(19) United States
(12) Patent Application Publication
(10) Pub. No.: US 2026/0285963 A1
(43) Pub. Date: Sep. 24, 2026

(54) ANTI-BCL6 NANOBODIES, FUSION PROTEINS AND USES THEREOF

(71) Applicant: Hubei University, Wuhan (CN)

(72) Inventors: Lixin MA, Wuhan (CN); Fei WANG, Wuhan (CN); Shuai ZHAO, Wuhan (CN); Yang WANG, Wuhan (CN); Fang YU, Wuhan (CN); Jingwei ZENG, Wuhan (CN); Wanping CHEN, Wuhan (CN)

(73) Assignee: Hubei University, Wuhan (CN)

(21) Appl. No.: 19/572,822

(22) Filed: Mar. 19, 2026

(30) Foreign Application Priority Data
Mar. 19, 2025 (CN) 2025103281679

(51) Int. Cl. C07K 16/22

(52) U.S. Cl. CPC C07K 16/22; C07K 2317/565; C07K 2317/567; C07K 2317/569; C07K 2319/02; C07K 2319/21; C07K 2319/70

(57) ABSTRACT

The present disclosure discloses anti-BCL6 nanobody, fusion protein comprising the same and use thereof, which belong to the field of biotechnology. In the present disclosure, artificial intelligence technology is employed to design nanobodies specifically targeting BCL6. The nanobodies exhibit favorable specificity and high affinity, and can efficiently bind to BCL6 antigen. Furthermore, rational design of the nanobodies is performed via artificial intelligence to obtain nanobody mutants with higher affinity. Furthermore, fusion proteins are obtained by fusing the nanobodies or nanobody mutants with high affinity and favorable specificity with an RBCC domain or a mutant thereof. The fusion proteins can specifically bind to the target protein via their nanobody domains, activate the protein degradation pathway, and achieve targeted degradation of BCL6 protein, thereby upregulating the intracellular expression of the tumor suppressor gene p53 and cyclin p21, which has great clinical application value.

### CROSS-REFERENCE TO RELATED APPLICATIONS

The application claims priority to Chinese patent application No. 2025103281679, filed on Mar. 19, 2025, the entire contents of which are incorporated herein by reference.

### SEQUENCE LISTING

The sequence listing xml file submitted herewith, named “Sequence_Listing.xml”, created on Mar. 19, 2026, and having a file size of 48,337 bytes, is incorporated by reference herein.

### TECHNICAL FIELD

The present disclosure belongs to the field of biotechnology, and specifically relates to anti-BCL6 nanobodies, fusion proteins and uses thereof.

### BACKGROUND

B-cell lymphoma 6 (BCL6) gene was originally identified in diffuse large B-cell lymphoma (DLBCL) and is one of the genes affected by chromosomal translocations. BCL6 is a transcriptional repressor belonging to the BTB/POZ family, consisting of an N-terminal BTB/POZ domain (bric-a-brac, tramtrack, broad complex, BTB), a middle PEST motif (also known as repression domain 2, RD2), and six C-terminal zinc finger domains (ZF). These domains regulate the transcription of target genes through diverse interactions. BCL6 mainly functions in the immune system and modulates the differentiation and maturation of B cells. During the germinal center (GC) reaction, BCL6 represses gene transcription to protect B cells from adverse factors such as somatic hypermutation and oxidative stress. It maintains genomic instability primarily by suppressing the expression of genes involved in DNA damage sensing or downstream checkpoint pathways, thereby promoting rapid proliferation and differentiation of B cells.

The germinal center is the primary site for B-cell differentiation and antibody affinity maturation. In the GC reaction, BCL6 helps B cells tolerate somatic hypermutation and oxidative stress generated during rapid proliferation by repressing DNA damage response-related genes, thus sustaining the normal progression of the GC reaction. This repressive function makes BCL6 critical for the generation of high-affinity B cells, effector memory B cells, and plasma cell differentiation. Dysregulated BCL6 expression may lead to abnormal GC reactions and further induce various lymphomas and other malignancies. Besides lymphomas, BCL6 also plays important roles in breast cancer, lung cancer and glioma, acting as a key regulator in cancer development and progression by modulating the expression of distinct target genes. Therefore, BCL6 represents an important target for cancer therapy.

Existing BCL6 inhibitors are mainly designed against its BTB domain and exert inhibitory activity by competitive binding with corepressors. However, research on BCL6 degraders is relatively limited. Among them, BCL6-targeting PROTACs (Proteolysis Targeting Chimeras) exhibit poor protein degradation activity, while other degraders are BCL6 molecular glues, which were accidentally discovered during inhibitor screening and are not amenable to rational design. These molecular glues show superior efficacy compared with similar inhibitors.

Nanobodies are single-domain antibodies derived from heavy-chain antibodies of camelids. They possess the specificity and affinity of conventional antibodies, while exhibiting smaller molecular weight and higher stability. Nanobodies can penetrate tissues and cells and bind target proteins more readily, thus showing broad application prospects in biomedical research and therapy.

Targeted protein degradation (TPD) technology utilizes the intracellular ubiquitin-proteasome system and lysosomal degradation system to achieve specific and efficient degradation of disease-related proteins, thereby achieving therapeutic effects. Compared with traditional small-molecule inhibitors, TPD has multiple advantages: it is event-driven, requires low dosage (only catalytic amounts are sufficient), and can target some traditionally undruggable proteins.

TRIM away is an emerging TPD technology that employs members of the TRIM (Tripartite motif-containing) protein family as E3 ubiquitin ligases to specifically recognize and bind target proteins, promote their ubiquitination, and ultimately lead to their degradation by the proteasome. The application of TRIM away technology in BCL6 degradation holds great potential and may provide a more efficient and precise approach for BCL6 degradation, thus expected to improve the therapeutic efficacy of BCL6-related cancers.

However, to date, no research on nanobodies targeting BCL6 has been reported.

### SUMMARY

In the prior art, there is a lack of nanobodies with high specificity, high affinity and the ability to efficiently bind to BCL6 antigen. The present disclosure aims to solve this problem.

In a first aspect, the present disclosure provides an anti-BCL6 protein nanobody, comprising complementarity-determining region 1 (CDR1), complementarity-determining region 2 (CDR2) and complementarity-determining region 3 (CDR3); wherein the CDR1 has an amino acid sequence shown in any one of SEQ ID NO: 1-2, the CDR2 has an amino acid sequence shown in any one of SEQ ID NO: 3-4, and the CDR3 has an amino acid sequence shown in any one of SEQ ID NO: 5-8.

In the present disclosure, the term “nanobody”, also known as single-domain antibody (sdAb), refers to an antibody consisting of only one V region of the heavy chain, and may also be referred to as VHH antibody. The antigen-binding ability and stability of nanobodies are basically consistent with those of intact antibodies. A nanobody is composed sequentially of frame region 1 (FR1), CDR1, frame region 2 (FR2), CDR2, frame region 3 (FR3), CDR3, and frame region 4 (FR4). A nanobody may be truncated at the N-terminus or C-terminus such that it comprises only a portion of FR1 and/or FR4, or lacks one or two of the framework regions, so long as it substantially retains antigen-binding activity and specificity. They are mainly responsible for antigen binding.

In some embodiments, the nanobody further comprises FR1, FR2, FR3, and FR4; wherein FR1 has an amino acid sequence shown in any one of SEQ ID NO: 9-10, FR2 has an amino acid sequence shown in any one of SEQ ID NO: 11-12, FR3 has an amino acid sequence shown in any one of SEQ ID NO: 13-14, and FR4 has an amino acid sequence shown in any one of SEQ ID NO: 15-16.

It is known that the framework regions are highly conserved. Therefore, conventional framework regions in the prior art can be selected according to actual application needs, so long as they can improve the stability of the nanobody.

In some embodiments, the nanobody is selected from any one of the following: A1) an amino acid sequence shown in any one of SEQ ID NO: 17-20; A2) an amino acid sequence having one or several amino acid substitutions, deletions or additions compared with the amino acid sequence defined in A1); A3) an amino acid sequence having a sequence identity of more than 80% compared with the amino acid sequence defined in A1) or A2); A4) an amino acid sequence obtained by linking a tag or a signal peptide to the N-terminus and/or C-terminus of the amino acid sequence defined in A1), A2) or A3).

In the present disclosure, the nanobodies specifically targeting BCL6 are designed using artificial intelligence technology. Such nanobodies possess favorable specificity and high affinity, and can efficiently bind to the BCL6 antigen.

In a second aspect, the present disclosure provides a mutant anti-BCL6 protein nanobody, wherein the nanobody mutants is selected from any one of the following: B1) an amino acid sequence shown in SEQ ID NO: 21; B2) an amino acid sequence having one or several amino acid substitutions, deletions or additions compared with the amino acid sequence defined in B1); B3) an amino acid sequence having a sequence identity of more than 80% compared with the amino acid sequence defined in B1) or B2); B4) an amino acid sequence derived by fusing a tag or a signal peptide to the N-terminus and/or C-terminus of the amino acid sequence defined in B1), B2) or B3); wherein in B1), The amino acid sequence as set forth in SEQ ID NO: 21 is derived from the amino acid sequence shown in SEQ ID NO: 18 by mutation at the following site: T106Q.

In the present disclosure, rational design of the nanobody using artificial intelligence is performed to obtain nanobody mutants with higher affinity.

In some embodiments, in addition to comprising the mutation at position 106 in the described nanobody mutants, may further contain conservative amino acid substitutions at other positions, such that the mutated nanobody exhibits higher affinity than the nanobody shown in SEQ ID NO: 18. Preferably, the conservative amino acid substitutions retain the high affinity of the nanobody of the present disclosure. It will be apparent to those skilled in the art that such substitutions can occur in regions other than the above-mentioned sites while retaining the corresponding affinity. Preferably, the conservative substitution variant has a conservative amino acid substitution at at least one position. Embodiments of conservative substitutions include substitutions within the following groups of amino acids: basic amino acids (such as arginine, lysine, and histidine), acidic amino acids (such as glutamic acid and aspartic acid), polar amino acids (such as glutamine and asparagine), hydrophobic amino acids (such as leucine, isoleucine, and valine), aromatic amino acids (such as phenylalanine, tryptophan, and tyrosine), and small amino acids (such as glycine, alanine, serine, threonine, and methionine).

In a third aspect, the present disclosure provides a fusion protein obtained by linking any one of the above nanobodies or the mutant nanobody to an RBCC domain or its mutant via a second linker;
wherein the amino acid sequence of the second linker is shown in SEQ ID NO: 31, the amino acid sequence of the RBCC domain is shown in SEQ ID NO: 29, and the amino acid sequence of the RBCC domain mutant is shown in SEQ ID NO: 30.

In the present disclosure, the nanobodies or nanobody mutants were fused with high affinity and favorable specificity to the RBCC domain or a mutant thereof (a truncated TRIM family protein) to obtain a fusion protein. By specifically binding to the target protein via its nanobody domain and activating the protein degradation pathway, the fusion protein can achieve targeted degradation of BCL6 protein, upregulate the intracellular expression of the tumor suppressor p53 and the cell cycle regulator p21, thereby protecting B cells from cell death induced by DNA damage and inhibiting the proliferation rate of cancer cells, which exhibits great clinical application value.

It is known that the linker peptide can be selected from conventional linker peptides in the prior art according to actual application needs, provided that it can facilitate good fusion of the two proteins.

In some embodiments, the fusion protein is selected from any one of the following: C1) having an amino acid sequence shown in any one of SEQ ID NOs: 35-44; C2) an amino acid sequence having one or several amino acid substitutions, deletions or additions compared with the amino acid sequence defined in C1); C3) an amino acid sequence having more than 80% sequence identity compared with the amino acid sequence defined in C1) or C2); C4) an amino acid sequence obtained by linking a tag or signal peptide to the N-terminus and/or C-terminus of the amino acid sequence defined in C1), C2) or C3).

The above-mentioned nanobodies, nanobody mutants, and fusion proteins provided by the present disclosure may be natural, recombinant or synthetic active polypeptides, which may be naturally purified products, chemically synthesized products, or products produced from prokaryotic hosts (such as Escherichia coli) or eukaryotic hosts (such as yeast, higher plants) using recombinant technology.

In A4), B4) or C4) of the present disclosure, the linkage may be direct linkage via peptide bonds or linkage via a linker, and the linkage method is a conventional method in the art. Among them, the tags include but are not limited to: GST (Glutathione S-transferase) tag protein, Trx (Thioredoxin) tag protein, His tag protein (His-tag), Flag tag protein, LacZ tag protein, GFP (Green Fluorescent Protein), sfGFP (Superfolder Green Fluorescent Protein), and HA tag (Hemagglutinin tag). Those skilled in the art can select an appropriate tag protein according to actual application needs. The use of the tag does not change the function of the target protein (nanobody, nanobody mutants or fusion protein), and its purpose is separation, purification, detection or tracing. The tag can be separated from the target protein (nanobody, nanobody mutants or fusion protein) by chemical cleavage or enzymatic methods known in the art (such as introducing a protease cleavage site to remove the tag by cleavage with TEV protease).

In a fourth aspect, the present disclosure provides a biomaterial selected from any one of the following: D1) a nucleic acid molecule encoding any one of the above-mentioned nanobodies, the above-mentioned nanobody mutants, or any one of the above-mentioned fusion proteins; D2) a recombinant vector containing the nucleic acid molecule of D1); D3) a recombinant cell containing the nucleic acid molecule of D1) or the recombinant vector of D2).

In some embodiments, the nucleic acid molecule defined in D1) is selected from any one of the following: E1) a nucleic acid molecule having a nucleotide sequence shown in any one of SEQ ID NOs: 22-26; E2) a nucleic acid molecule that hybridizes with the nucleic acid molecule defined in E1) under stringent conditions and encodes any one of the above-mentioned nanobodies, the above-mentioned nanobody mutants, or any one of the above-mentioned fusion proteins; E3) a nucleic acid molecule having more than 90% sequence identity with the nucleic acid molecule defined in E1) or E2) and encoding any one of the above-mentioned nanobodies, the above-mentioned nanobody mutants, or any one of the above-mentioned fusion proteins.

The above-mentioned nucleic acid molecules provided by the present disclosure may be DNA, such as cDNA, genomic DNA or recombinant DNA; or RNA, such as mRNA or hnRNA; and the nucleic acid molecules can usually be obtained by PCR amplification or artificial synthesis.

The above-mentioned recombinant vectors provided by the present disclosure include cloning vectors and expression vectors. Cloning vectors are used for replicating related sequences, and expression vectors are used for expressing related genes. Among them, the vectors used in constructing the expression vectors may be at least one of pET23a, pET28a, and pcDNA3.1 vectors.

In some embodiments, the preparation method of the above-mentioned recombinant cells includes the step of transforming the above-mentioned recombinant vector into an expression host cell.

In the present disclosure, the expression host cell is a conventional host cell in the art, as long as it can ensure that the recombinant vector can stably replicate on its own and the gene carried thereby can be effectively expressed. It can be a prokaryotic cell or a eukaryotic cell, such as Escherichia coli, yeast, etc. Embodiments of Escherichia coli include E. coli BL21 (DE3), Rosetta (DE3), BL21 (DE3) plysS. In the present disclosure, the Escherichia coli expression host E. coli BL21 (DE3) is preferred.

In a fifth aspect, the present disclosure provides the use of any one of the above-mentioned nanobodies, the above-mentioned nanobody mutants, any one of the above-mentioned fusion proteins, or the above-mentioned biomaterial in any one of the following: F1) Use in the preparation of products for preventing and/or treating BCL6 target-related diseases; F2) Use in the preparation of products for screening, diagnosing or assisting in diagnosing BCL6 target-related diseases; wherein, the BCL6 target-related diseases are BCL6-positive tumors.

In the present disclosure, BCL6-positive tumors refer to tumors in which BCL6 expression is detected in tumor cells. Such tumors may include common tumors such as follicular lymphoma, diffuse large B-cell lymphoma, Burkitt lymphoma, and Hodgkin lymphoma.

In a sixth aspect, the present disclosure provides a pharmaceutical composition for preventing and/or treating BCL6 target-related diseases, the pharmaceutical composition comprising any one of the above-mentioned nanobodies, the above-mentioned nanobody mutants, any one of the above-mentioned fusion proteins, or the above-mentioned biomaterial, and a pharmaceutically acceptable carrier.

In the present disclosure, the term “pharmaceutically acceptable carrier” refers to excipients widely used in the field of pharmaceutical production. Excipients are mainly used to provide a safe, stable and functional pharmaceutical composition, and can also provide a method to make the active ingredient dissolve at a desired rate after administration to the subject, or promote the effective absorption of the active ingredient after the subject receives the composition. Pharmaceutical excipients may be inert fillers, or provide a certain function, such as stabilizing the overall pH value of the composition or preventing the degradation of the active ingredient of the composition.

The pharmaceutical composition provided by the present disclosure can be prepared by any method known to those skilled in the art according to the disclosed content. For Embodiment, it includes but is not limited to conventional mixing, dissolving, granulating, emulsifying, grinding, encapsulating, embedding or lyophilizing processes.

In some embodiments, the dosage form of the pharmaceutical composition includes at least one of solid preparations, semi-solid preparations, and liquid preparations.

The pharmaceutical composition provided by the present disclosure can be administered in any form, including injection (intravenous), mucosal, oral (solid and liquid preparations), inhalation, ophthalmic, rectal, topical or parenteral (infusion, injection, implantation, subcutaneous, intravenous, intraarterial, intramuscular) administration. The pharmaceutical composition of the present disclosure can also be a controlled-release or sustained-release dosage form (such as liposomes or microspheres).

In a seventh aspect, the present disclosure provides a method for preparing any one of the above-mentioned nanobodies, the above-mentioned nanobody mutants, or any one of the above-mentioned fusion proteins, comprising the following steps: culturing the above-mentioned recombinant cells, and obtaining a culture after induced expression; isolating the nanobody, nanobody mutants or fusion protein from the culture.

In the present disclosure, there are no special requirements on the culture method, culture conditions and culture medium, as long as the normal growth of the recombinant cells is ensured. Moreover, the methods for isolating any one of the above-mentioned nanobodies, the above-mentioned nanobody mutants, or any one of the above-mentioned fusion proteins from the culture are all conventional methods in the art.

The advantageous effects of the present disclosure are: different from the prior art, the present disclosure designs nanobodies specifically targeting BCL6 by using artificial intelligence technology. The nanobodies have good specificity and high affinity, and can efficiently bind to BCL6 antigen; further, rational design of nanobodies is performed by artificial intelligence to obtain nanobody mutants with higher affinity; further, fusion proteins are obtained by fusing nanobodies or nanobody mutants with high affinity and good specificity with RBCC domain or its mutants. The fusion proteins can specifically bind to the target protein through their nanobody domains, trigger the protein degradation pathway, achieve targeted degradation of BCL6 protein, increase the intracellular expression of the tumor suppressor gene p53 and the cell cycle protein p21, thereby protecting B cells from cell death induced by DNA damage and inhibiting the proliferation rate of cancer cells, which has great clinical application value.

### BRIEF DESCRIPTION OF DRAWINGS

FIG. 1 is a schematic diagram of the element arrangement of the recombinant expression vector constructed in the present disclosure;

FIG. 2 shows the SDS-PAGE detection results of nanobodies in Example 2, wherein in panel (a), lane M is Marker, and lanes 0-6 are sfGFP-BN0 to sfGFP-BN6, respectively; in panel (b), lane M is Marker, and lanes 0-6 are sfGFP-BNC0 to sfGFP-BNC6, respectively;

FIG. 3 shows the SDS-PAGE detection results of BCL6 antigen in Example 2;

FIG. 4 shows the ELISA test results of the affinity between nanobodies and BCL6 antigen in Example 3;

FIG. 5 shows the BLI test results of the affinity between nanobodies BN1, BN2, BN4, BNC2 and BCL6 antigen in Example 4;

FIG. 6 shows the binding energy and total score of BN2-WT and mutant BN2M with BCL6 antigen calculated by Rosetta (panels a and b) and Amber (panel c) in Example 5, respectively;

FIG. 7 shows the energy changes before and after the T106Q mutation in mutant BN2M calculated by Amber (panels a and b) and the ELISA test results of the affinity between nanobody mutants and BCL6 antigen (panels c and d) in Example 5, respectively;

FIG. 8 shows the results of GFP plasmid electroporation into Raji cells in Example 6;

FIG. 9 shows the WB results of targeted degradation of BCL6 protein by fusion proteins in Example 6, wherein 1 is RBCC-BN1, 2 is RBCC-BN2M, 3 is RBCC-BN2, 4 is RBCC-BNC2, and 5 is the control;

FIG. 10 shows the WB results of targeted degradation of BCL6 protein by fusion proteins in Example 6, wherein 1 is RS80E-BN1, 2 is RS80E-BN2M, 3 is RS80E-BN2, 4 is RS80E-BNC2, and 5 is the control;

FIG. 11 shows the WB results of targeted degradation of BCL6 protein by fusion protein RS80E-BNC2 in Example 6;

FIG. 12 shows the changes in p53 and p21 levels during BCL6 protein degradation by fusion proteins RBCC-BN2 and RS80E-BNC2 in Example 6, wherein a and b correspond to RBCC-BN2, and c and d correspond to RS80E-BNC2;

FIG. 13 shows the WB results verifying the pathway of targeted degradation of BCL6 protein by fusion proteins in Example 6, wherein 1 is the control, 2 is RBCC-BNC2, 3 is RBCC-BNC2+MG132, and 4 is RBCC-BNC2+BafA1.

### DETAILED DESCRIPTION OF THE EMBODIMENTS

Hereinafter, the technical solutions in the Embodiments of the present disclosure will be clearly and completely described in conjunction with the Embodiments of the present disclosure. Obviously, the described Embodiments are only a part of the Embodiments of the present disclosure, rather than all the Embodiments. Based on the Embodiments of the present disclosure, all other Embodiments obtained by those of ordinary skill in the art without making creative efforts shall fall within the protection scope of the present disclosure.

For the experimental methods without specifying specific conditions in the Embodiments, they are usually carried out in accordance with conventional experimental methods in the field of molecular biology, including but not limited to the experimental methods described in “Molecular Cloning: A Laboratory Manual” by M. R. Green and “Molecular Biology” by Robert⋅F⋅Weaver, or in accordance with the experimental methods recommended by the kit and instrument manufacturers. Unless otherwise specified, the reagents and biomaterials used in the Embodiments can be obtained from commercial channels.

### Example 1 Artificial Intelligence Design of Nanobodies

First, artificial intelligence technology combined with bioinformatics tools (such as SEPPA, MetMHCpan, etc.) is used to predict hot-spot epitopes of the BCL6 antigen, and linear fragmentation is performed thereon to generate fragments comprising at least four consecutive residues. Subsequently, CDR-like fragments matching the above fragments are screened from the AbAg database, and structural superposition and optimization are performed between the CDR-like fragments and the epitope fragments to construct CDR fragments capable of specifically binding the BCL6 antigen. Appropriate fragments are then selected (the amino acid sequences of the CDR fragments and fragments are shown in Table 1 below). Finally, a series of nanobodies targeting specific epitopes of the BCL6 antigen are obtained, which are named nanobodies BN1-6 and BNC1-6, respectively.

TABLE 1
Amino acid sequences of CDR
fragments and fragments

| Fragment | Amino acid sequence |
|---|---|
| CDR1 | GFNIKDT |
| | (SEQ ID NO: 1) |
| | SGGSEYSYSTF |
| | (SEQ ID NO: 2) |
| CDR2 | YPTSGY |
| | (SEQ ID NO: 3) |
| | ASMGGL |
| | (SEQ ID NO: 4) |
| CDR3 | GSGITTIKEMGRSIHEIPREEFDY |
| | (SEQ ID NO: 5) |
| | GSGLVATVKEAGRSIHEIPREELEEFDY |
| | (SEQ ID NO: 6) |
| | GSVGASTLYGSAYEEFDY |
| | (SEQ ID NO: 7) |
| | GLVATVKEAGRSIHEIPREEL |
| | (SEQ ID NO: 8) |
| FR1 | MEVQLEESGGGLVQPGGSLRLSCAAS |
| | (SEQ ID NO: 9) |
| | EFQVQLVESGGGSVQAGGSLRLSCTAS |
| | (SEQ ID NO: 10) |
| FR2 | YIGWVRQAPGKGEEWVASI |
| | (SEQ ID NO: 11) |
| | SLGWFRQAPGQEREAVAAI |
| | (SEQ ID NO: 12) |
| FR3 | TRYADSVKGRFTISADTSKNTAYLQM |
| | NSLRAEDTAVYYCAA |
| | (SEQ ID NO: 13) |
| | TYYADSVKGRFTISRDNAKNTVTLQM |
| | NNLKPEDTAIYYCAA |
| | (SEQ ID NO: 14) |
| FR4 | WGQGTLVTVSS |
| | (SEQ ID NO: 15) |
| | WGQGTQVTVSS |
| | (SEQ ID NO: 16) |

Exemplarily, the amino acid sequence of nanobody BN1 is shown below:

(SEQ ID NO: 17)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSGITTIKEMGRSIHEIPREEFDYWGQGTLVTVSS;

The underlined regions represent CDR1, CDR2, and CDR3 in sequence.

The amino acid sequence of nanobody BN2 is shown below:

(SEQ ID NO: 18)
MEVQLEESGGGLVQPGGSLRLSCAASGFNIKDTYIGWVRQAPGKGE
EWVASIYPTSGYTRYADSVKGRFTISADTSKNTAYLQMNSLRAED
TAVYYCAAGSGLVATVKEAGRSIHEIPREELEEFDYWGQGTLVTV
SS;

The underlined regions represent CDR1, CDR2, and CDR3 in sequence.

The amino acid sequence of nanobody BN4 is shown below:

(SEQ ID NO: 19)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSVGASTLYGSAYEEFDYWGQGTLVTVSS;

The underlined regions represent CDR1, CDR2, and CDR3 in sequence.

The amino acid sequence of nanobody BNC2 is shown below:

(SEQ ID NO: 20)
EFQVQLVESGGGSVQAGGSLRLSCTASGGSEYSYSTFSLGWFRQA
PGQEREAVAAIASMGGLTYYADSVKGRFTISRDNAKNTVTLQMNN
LKPEDTAIYYCAAGLVATVKEAGRSIHEIPREELWGQGTQVTVS
S;

The underlined regions represent CDR1, CDR2, and CDR3 in sequence.

The amino acid sequence of the nanobody BN0 framework is obtained by removing the CDR1, CDR2, and CDR3 from nanobody BN1 (the amino acid sequence of which is shown in SEQ ID NO: 17) (comprising only sequences).

The amino acid sequence of the nanobody BNC0 framework is obtained by removing the CDR1, CDR2, and CDR3 from nanobody BNC2 (the amino acid sequence of which is shown in SEQ ID NO: 20) (comprising only sequences).

The nucleotide sequences of the genes encoding nanobodies BN1, BN2, BN4, and BNC2 are shown in SEQ ID NOs: 22-25, respectively.

### Example 2 Expression and Purification of Nanobodies and BCL6 Antigen

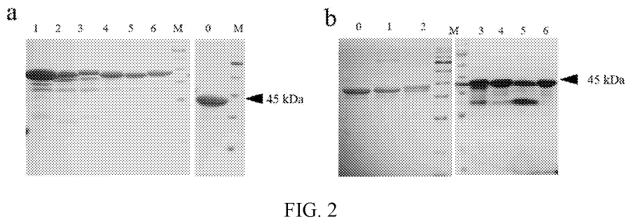

The constructed recombinant plasmids were respectively transformed into E. coli competent cells BL21 (DE3), and cultured statically overnight at 37° C. to obtain a series of recombinant strains. Single colonies were picked and inoculated into 100 mL LB liquid medium (ampicillin concentration: 50 μg/mL), followed by shaking culture at 37° C. When OD600 reached about 0.6, IPTG was added to a final concentration of 0.5 mM, and induction culture was performed with shaking at 18° C. for 18 hours. After completion of the culture, the cells and culture supernatant were collected by centrifugation at 12000 rpm and 4° C., respectively. The cells were washed with TEN buffer (50 mM Tris-HCl, 5 mM EDTA, 50 mM NaCl, pH 8.0) at 4° C. for more than 4 hours, and the protein secreted and expressed by the strain was directly obtained from the TEN buffer. SDS-PAGE was used to detect and analyze the secretory expression of the nanobodies, and the results are shown in FIG. 2.

It is showed in the FIG. 2 that nanobodies BN1-6 and BNC1-6 were all successfully expressed.

Exemplarily, the purified nanobodies BN1, BN2, BN4, and BNC2 obtained by outer membrane washing were preserved for subsequent experiments.

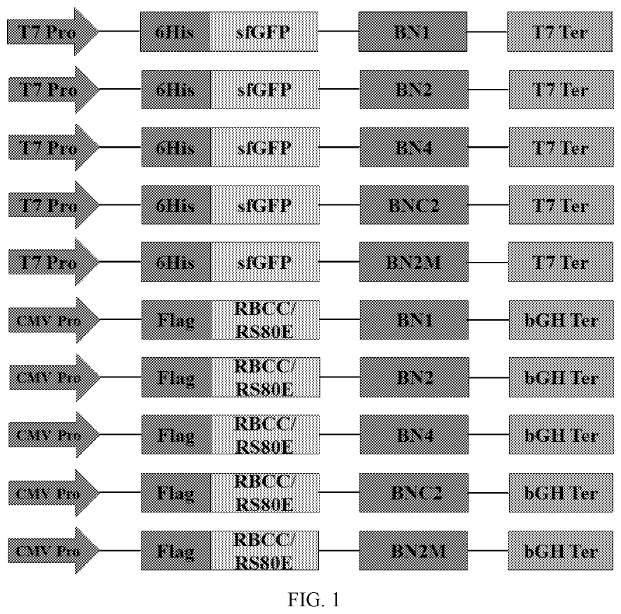

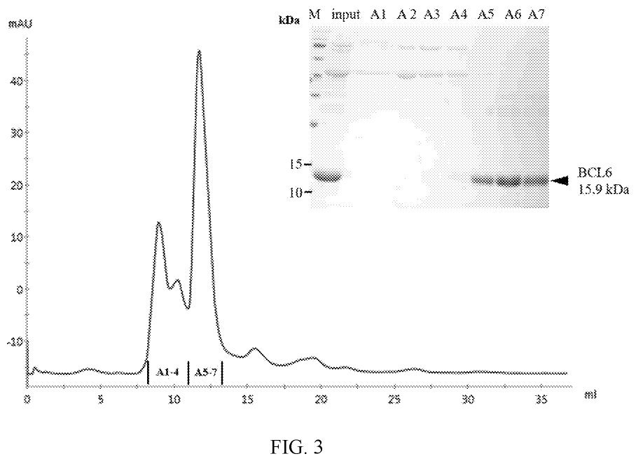

Furthermore, the BCL6 antigen sequence (amino acids 6-129 of NCBI Accession No. P41182) was cloned into the pET28a vector by homologous recombination to obtain the recombinant plasmid pET28a-BCL6 (vector structure shown in FIG. 1). The constructed recombinant plasmid was transformed into E. coli competent cells BL21 (DE3), respectively. Single colonies were picked and inoculated into 100 mL LB liquid medium (kanamycin concentration: 100 μg/mL), followed by static culture at 37° C. overnight. When OD600 reached about 0.6, IPTG was added to a final concentration of 0.5 mM, and induction culture was performed with shaking at 18° C. for 18 hours. The cells were collected by centrifugation at 6000 rpm for 10 min. After washing with Buffer A (20 mM Tris-HCl pH 8.0, 200 mM NaCl), the cells were resuspended in Buffer A, supplemented with PMSF to a final concentration of 1 mM, and disrupted under high pressure. After centrifugation at 18000 rpm for 30 min, the supernatant was collected, filtered, and subjected to Ni-NTA purification. The protein purified by Ni-NTA was further purified by size-exclusion chromatography (Superdex 75 increase 10/300, GL), which had been pre-equilibrated with Buffer A. The purified protein was collected and detected by SDS-PAGE, divided into aliquots, snap-frozen in liquid nitrogen, and stored at −80° C. The SDS-PAGE identification results are shown in FIG. 3. Calculated via http://www.expasy.org/, the expected size of BCL6 is 15.9 kDa. The results show that the BCL6 antigen was successfully expressed.

### Example 3 ELISA Assay of Nanobodies

This example is intended to verify whether the nanobodies BN1, BN2, BN4 and BNC2 purified in Example 2 can directly interact with the BCL6 antigen purified in Example 2. Specifically, the steps are as follows:
Dilute the BCL6 antigen to 1 μg/mL with 1×ELISA coating buffer, add 100 μL per well to coat the microplate, and incubate statically at 4° C. overnight;
Wash the plate with PBST, and block with 1% BSA at room temperature for 2 h with 100 μL per well;
Prepare nanobodies BN1, BN2, BN4 and BNC2 at different concentrations with 1% BSA, add 100 μL per well, and incubate at room temperature for 1 h;
Incubate with HRP-conjugated Mouse anti-HA-Tag mAb as the secondary antibody at room temperature for 1 h;
Develop color with TMB, and terminate the reaction with stop solution;
Measure the absorbance at 450 nm with a microplate reader.

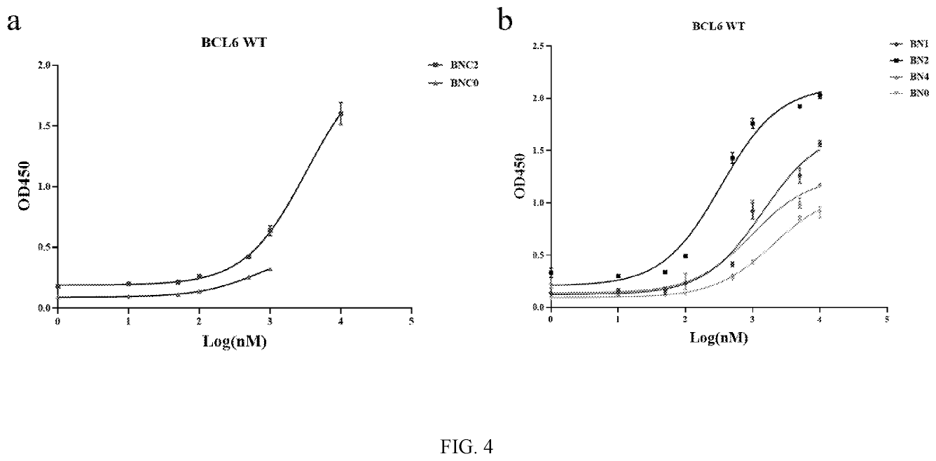

The test results are shown in FIG. 4.

It can be seen from FIG. 4 that the nanobodies BN1, BN2, BN4 and BNC2 exhibit favorable binding affinity with the BCL6 antigen.

### Example 4 Bio-Layer Interferometry (BLI) Assay of Nanobodies

This example is used to further verify the binding between the nanobodies BN1, BN2, BN4, BNC2 purified in Example 2 and the BCL6 antigen purified in Example 2, and to calculate the binding affinity therebetween.

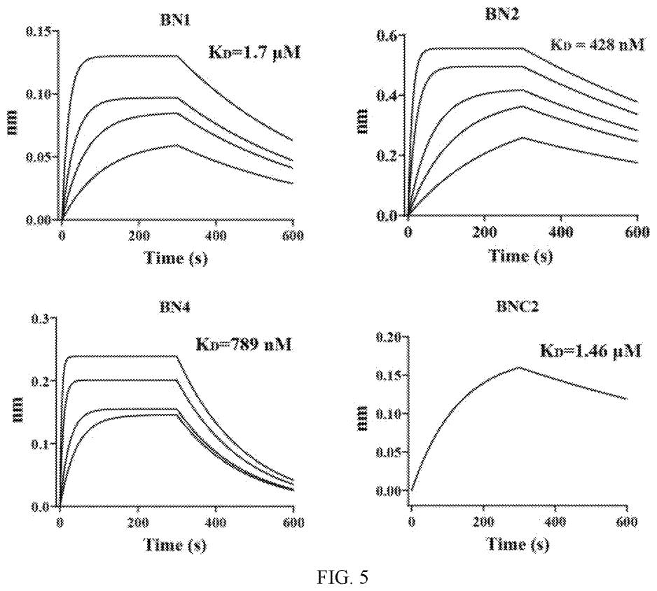

Specifically, the BCL6 antigen was immobilized on a biosensor chip, and the nanobodies BN1, BN2, BN4, BNC2 at different concentration gradients were sequentially added to analyze the binding affinity with the antigen protein. The increase in optical thickness at the tip of the biosensor within 600 seconds was recorded, and the binding affinity with BCL6 was calculated according to the association and dissociation rates of the antibodies at different concentrations. The results are shown in FIG. 5.

It can be seen from FIG. 5 that the nanobodies BN1, BN2, BN4 and BNC2 exhibit favorable binding affinity with the BCL6 antigen.

### Example 5 Affinity Improvement Assay of Nanobodies

To further improve the affinity of nanobodies, the present disclosure adopts artificial intelligence for rational design of nanobodies, and mutates nanobody BN2 to obtain the following mutants BN2M-T106Q (denoted as T106Q) and BN2M-T106E (denoted as T106E).

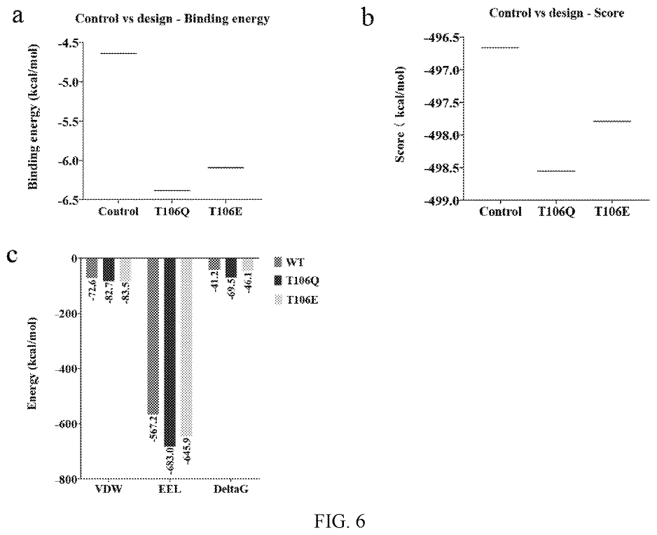

Furthermore, the binding energy and total score of BN2-WT and mutant BN2M with the BCL6 antigen were calculated by Rosetta, and then the energy changes of BN2-WT, T106Q and T106E were calculated by Amber. The results are shown in FIG. 6.

It can be seen from FIG. 6 that both T106Q and T106E have higher scores (FIG. 6b) and lower energy values (FIG. 6a), and T106Q has a lower energy value (FIG. 6c), which is expected to have lower affinity.

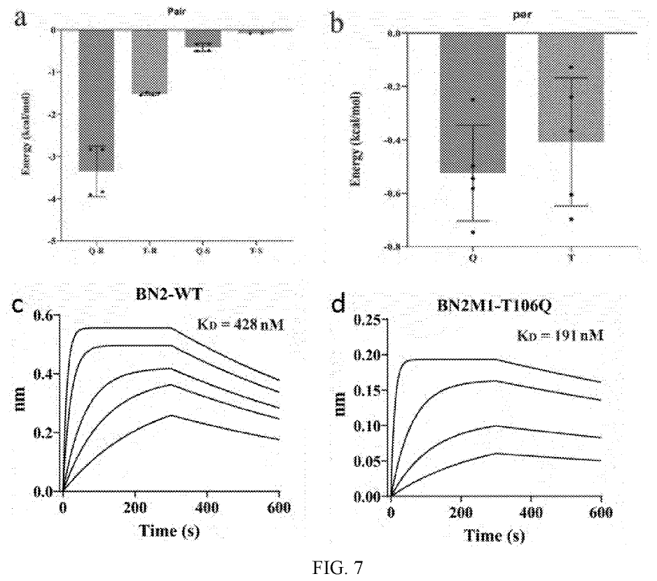

Furthermore, Amber was used to calculate the individual energy change of the BN2M-T106Q mutation site. It was found that after the 106th position of BN2M-T106Q was mutated from T to Q, the energy value decreased and the stability was better (FIG. 7b). For the complex pairing energy calculation, it was found that the overall paired amino acids of the mutated Q also had lower energy values compared with the pre-mutation T (FIG. 7a).

The expression vector of nanobody mutants BN2M was constructed by the method in Example 2 (see FIG. 1). Wherein, the nanobody mutants BN2M comprises BN2M-T106Q, and the nucleotide sequence of BN2M-T106Q is obtained by replacing the nucleotides “ACC” at positions 316-318 of SEQ ID NO: 18 with “CAG”.

Then induced expression and purification were performed to obtain the nanobody mutants BN2M-T106Q.

The affinity of the above BN2 and its mutant BN2M with the BCL6 antigen was detected by ELISA assay according to the method in Example 3, and the results are shown in FIGS. 7c-7d.

It can be seen from FIGS. 7c-7d that compared with the affinity of nanobody BN2 with the BCL6 antigen, BN2M-T106Q has better affinity with the BCL6 antigen, and the affinity is increased by about 2.2 folds.

Finally, the nanobody mutants BN2M-T106Q with significantly improved affinity was obtained and named nanobody mutants BN2M.

Wherein, the amino acid sequence of the nanobody mutants BN2M is shown below:

(SEQ ID NO: 21)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSGLVAQVKEAGRSIHEIPREELEEFDYWGQGTLVT
VSS;

The underlined regions represent CDR1, CDR2 and CDR3 in sequence.

The nucleotide sequence of the gene encoding the nanobody mutants BN2M is shown in SEQ ID NO: 26.

### Example 6 Targeted Degradation of BCL6 Protein by Fusion Proteins

To achieve targeted degradation of BCL6 protein, in the present disclosure, the above nanobodies BN1, BN2, BN4, BNC2 and nanobody mutants BN2M were fused with the RBCC domain or its mutant RS80E, respectively, to obtain fusion proteins. The fusion proteins were transfected into Raji human Burkitt's lymphoma cells, and the targeted degradation efficiency of BCL6 protein by the fusion proteins was detected, with BN0 framework containing only sequences as the control.

Specifically, using pcDNA3.1 as the base vector, the genes encoding the above nanobodies BN1, BN2, BN4, BNC2, nanobody mutants BN2M and BN0 framework were ligated and fused with the gene encoding the RBCC domain (its amino acid sequence is shown in SEQ ID NO: 29, and the nucleotide sequence is shown in SEQ ID NO: 32) or its mutant RS80E (its amino acid sequence is shown in SEQ ID NO: 30, and the nucleotide sequence is shown in SEQ ID NO: 33) via a second linker (its amino acid sequence is shown in SEQ ID NO: 31, and the nucleotide sequence is shown in SEQ ID NO: 34). Expression vectors were constructed by homologous recombination, and the element arrangement structure of the expression vectors is shown in FIG. 1. The recombinant plasmids were pcDNA3.1-RBCC-BN1, pcDNA3.1-RBCC-BN2, pcDNA3.1-RBCC-BN4, pcDNA3.1-RBCC-BNC2, pcDNA3.1-RBCC-BN2M, pcDNA3.1-RBCC-BN0, pcDNA3.1-RS80E-BN1, pcDNA3.1-RS80E-BN2, pcDNA3.1-RS80E-BN4, pcDNA3.1-RS80E-BNC2 and pcDNA3.1-RS80E-BN2M, respectively.

Wherein, the amino acid sequences of the fusion proteins RBCC-BN1, RBCC-BN2, RBCC-BN4, RBCC-BNC2, RBCC-BN2M, RS80E-BN1, RS80E-BN2, RS80E-BN4, RS80E-BNC2 and RS80E-BN2M are shown in SEQ ID NOs: 35-44, respectively.

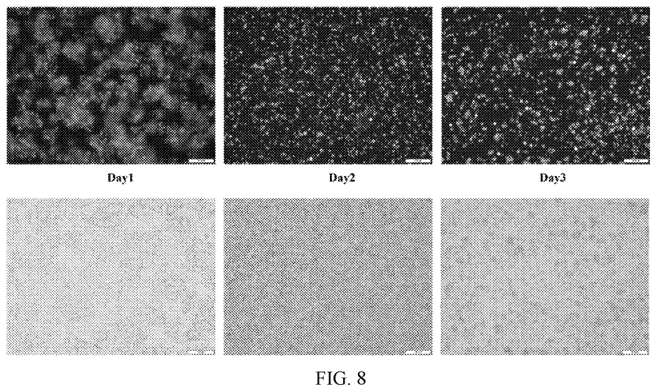

GFP plasmid was electroporated into well-growing Raji cells using a Lonza 4D electroporator. After electroporation, the electroporation efficiency and protein expression were observed via the GFP plasmid, and the results are shown in FIG. 8.

It can be seen from FIG. 8 that the above transfection method has high transfection efficiency and can achieve highly efficient plasmid transfection to the greatest extent.

Exemplarily, the recombinant plasmids pcDNA3.1-RBCC-BN1, pcDNA3.1-RBCC-BN2, pcDNA3.1-RBCC-BNC2, pcDNA3.1-RBCC-BN2M, pcDNA3.1-RBCC-BN0 and GFP plasmid were electroporated into well-growing Raji cells, respectively, according to the above method.

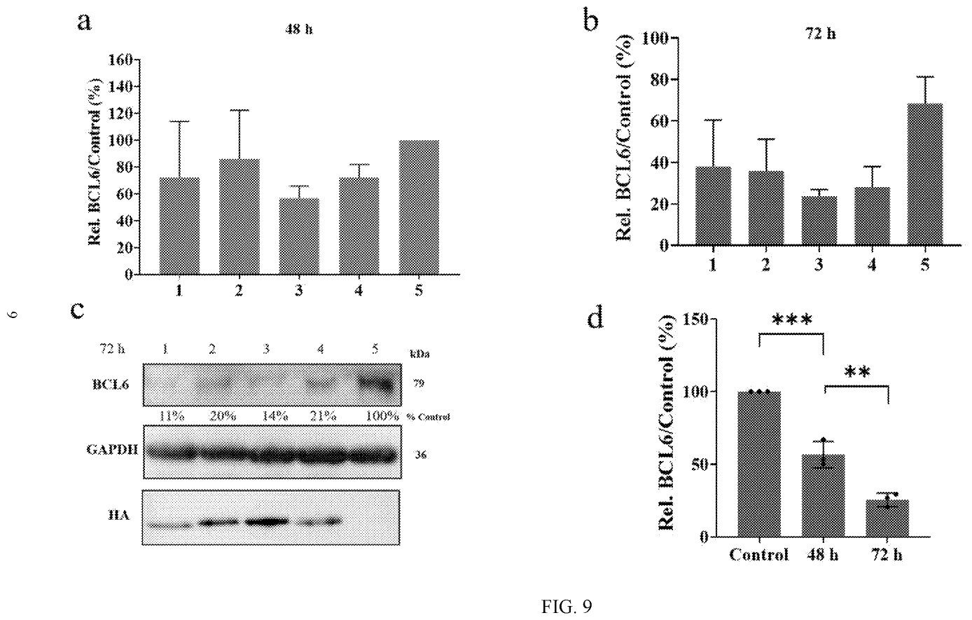

Cells were collected at 48 h and 72 h post-transfection, and then detected by Western blot, respectively. The targeted protein degradation efficiency at 48 h and 72 h post-transfection was calculated according to the Western blot results, and the results are shown in FIG. 9.

It can be seen from FIG. 9 that compared with the control, the fusion proteins RBCC-BN1, RBCC-BN2, RBCC-BN4, RBCC-BNC2 and RBCC-BN2M all exert a certain degradation effect on BCL6 protein, and the fusion protein RBCC-BN2 exhibits the most significant degradation effect on BCL6 protein, with a targeted protein degradation efficiency of about 50% at 48 h and about 70% at 72 h.

Similarly, exemplarily, the recombinant plasmids pcDNA3.1-RS80E-BN1, pcDNA3.1-RS80E-BN2, pcDNA3.1-RS80E-BNC2, pcDNA3.1-RS80E-BN2M, pcDNA3.1-RBCC-BN0 and GFP plasmid were electroporated into well-growing Raji cells, respectively, according to the above method.

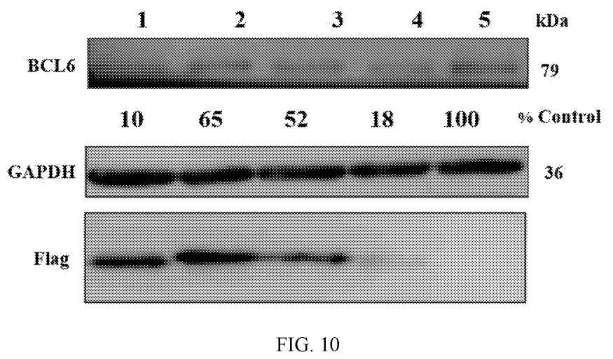

Cells were collected at 72 h post-electroporation and detected by Western blot, respectively. The targeted protein degradation efficiency at 72 h post-transfection was calculated according to the Western blot results, and the results are shown in FIG. 10.

It can be seen from FIG. 10 that the fusion protein RS80E-BN2 exhibits the most significant and stable degradation effect after electroporation.

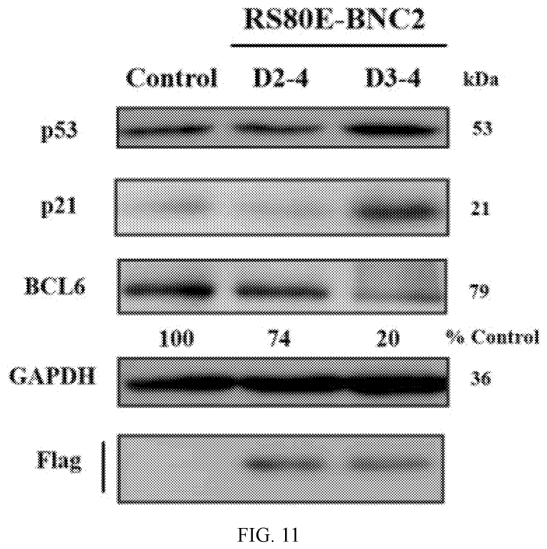

Furthermore, cells were collected at 48 h and 72 h after electroporation with the recombinant plasmid pcDNA3.1-RS80E-BNC2 and detected by Western blot, respectively. The targeted protein degradation efficiency at 48 h and 72 h post-transfection was calculated according to the Western blot results, and the results are shown in FIG. 11.

It can be seen from FIG. 11 that the fusion protein RS80E-BNC2 exhibits the most significant effect after electroporation, with a targeted protein degradation efficiency of about 40% at 48 h (lanes D2-4) and about 80% at 72 h (lanes D3-4).

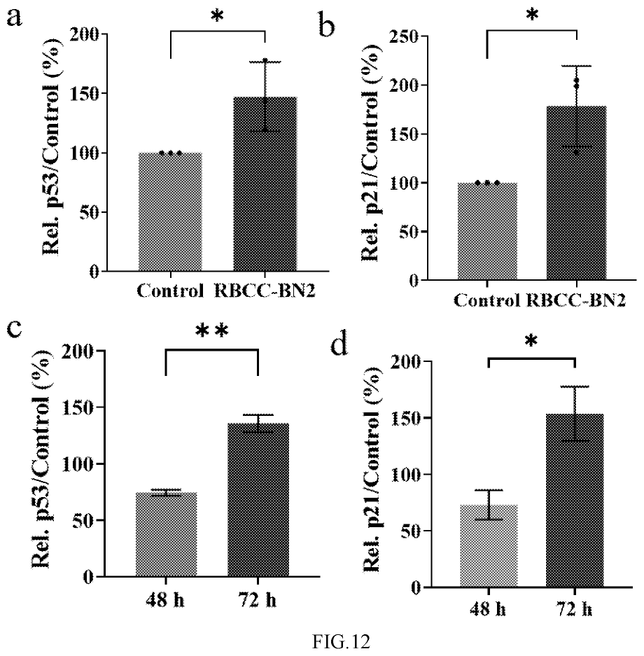

Furthermore, cells were collected after electroporation with the recombinant plasmids pcDNA3.1-RS80E-BNC2 and pcDNA3.1-RBCC-BN2 and detected by Western blot, respectively. The expression levels of the tumor suppressor gene p53 and cyclin p21 in the cells were obtained according to the Western blot results, and the results are shown in FIG. 12.

It can be seen from FIG. 12 that the degradation of BCL6 protein by the fusion proteins RS80E-BNC2 and RBCC-BN2 is accompanied by the up-regulation of the intracellular expression levels of the tumor suppressor gene p53 and cyclin p21.

Finally, the degradation pathway of targeted degradation of BCL6 protein by the fusion proteins was investigated.

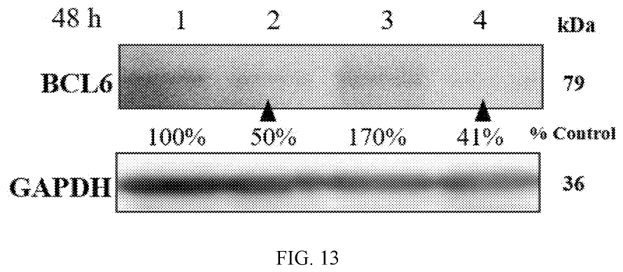

Specifically, MG132 proteasome inhibitor at a final concentration of 10 μM or BafA1 autophagy inhibitor at a final concentration of 100 nM was used, wherein MG132 can inhibit the ubiquitin-proteasome pathway and BafA1 can inhibit the lysosomal pathway. Three samples of pcDNA3.1-RBCC-BNC2 recombinant plasmid and GFP plasmid were electroporated into well-growing Raji cells, respectively. At 24 h post-transfection, corresponding MG132 and BafA1 were added to two of the samples of cells transfected with pcDNA3.1-RBCC-BNC2 recombinant plasmid, and Western blot detection was performed after further culture. The results are shown in FIG. 13.

It can be seen from FIG. 13 that the targeted degradation of BCL6 protein by the fusion proteins in the present disclosure is mediated via the ubiquitin-proteasome pathway.

In summary, the present disclosure designs nanobodies specifically targeting BCL6 by using artificial intelligence technology. The nanobodies have favorable specificity and high affinity, and can efficiently bind to BCL6 antigen. Furthermore, rational design of nanobodies is performed via artificial intelligence to obtain nanobody mutants with higher affinity. Further, fusion proteins are obtained by fusing nanobodies or nanobody mutants with high affinity and favorable specificity with the RBCC domain or its mutant thereof. The fusion proteins can specifically bind to the target protein via their nanobody domains, activate the protein degradation pathway, and achieve targeted degradation of BCL6 protein.

The above-described examples only express the implementation modes of the present disclosure, and the descriptions thereof are relatively specific and detailed, but shall not be construed as limiting the scope of the disclosure patent. It should be noted that, for those of ordinary skill in the art, several modifications and improvements can be made without departing from the concept of the present disclosure, and these all fall within the protection scope of the present disclosure. Therefore, the protection scope of the disclosure patent shall be subject to the appended claims.

What is claimed is:

1. An anti-BCL6 protein nanobody, comprising complementarity-determining region 1 (CDR1), complementarity-determining region 2 (CDR2) and complementarity-determining region 3 (CDR3);
wherein the amino acid sequence of CDR1 is shown in SEQ ID NO: 1, the amino acid sequence of CDR2 is shown in SEQ ID NO: 3, and the amino acid sequence of CDR3 is shown in SEQ ID NO: 5;
or the amino acid sequence of CDR1 is shown in SEQ ID NO: 1, the amino acid sequence of CDR2 is shown in SEQ ID NO: 3, and the amino acid sequence of CDR3 is shown in SEQ ID NO: 6;
or the amino acid sequence of CDR1 is shown in SEQ ID NO: 1, the amino acid sequence of CDR2 is shown in SEQ ID NO: 3, and the amino acid sequence of CDR3 is shown in SEQ ID NO: 7;
or the amino acid sequence of CDR1 is shown in SEQ ID NO: 2, the amino acid sequence of CDR2 is shown in SEQ ID NO: 4, and the amino acid sequence of CDR3 is shown in SEQ ID NO: 8.

2. The anti-BCL6 protein nanobody according to claim 1, wherein the nanobody further comprises framework region 1 (FR1), framework region 2 (FR2), framework region 3 (FR3) and framework region 4 (FR4);
wherein the amino acid sequence of FR1 is shown in SEQ ID NO: 9, the amino acid sequence of FR2 is shown in SEQ ID NO: 11, the amino acid sequence of FR3 is shown in SEQ ID NO: 13, and the amino acid sequence of FR4 is shown in SEQ ID NO: 15;
or the amino acid sequence of FR1 is shown in SEQ ID NO: 10, the amino acid sequence of FR2 is shown in SEQ ID NO: 12, the amino acid sequence of FR3 is shown in SEQ ID NO: 14, and the amino acid sequence of FR4 is shown in SEQ ID NO: 16.

3. The anti-BCL6 protein nanobody according to claim 2, wherein the nanobody is selected from any one of the following:
A1) the amino acid sequence is shown in any one of SEQ ID NOs: 17-20;
A2) an amino acid sequence obtained by linking a tag or a signal peptide to the N-terminus and/or C-terminus of the amino acid sequence defined in A1).

4. A mutant of an anti-BCL6 protein nanobody, wherein the mutant nanobody is selected from any one of the following:
B1) the amino acid sequence is shown in SEQ ID NO: 21;
B2) an amino acid sequence obtained by linking a tag or a signal peptide to the N-terminus and/or C-terminus of the amino acid sequence defined in B1).

5. A fusion protein, wherein the fusion protein is obtained by linking the nanobody mutants according to claim 4 with an RBCC domain or a mutant thereof via a second linker;
wherein the amino acid sequence of the second linker is shown in SEQ ID NO: 31, the amino acid sequence of the RBCC domain is shown in SEQ ID NO: 29, the amino acid sequence of the RBCC domain mutant is shown in SEQ ID NO: 30; and the amino acid sequence of the fusion protein is shown in any one of SEQ ID NOs: 35, 36, 38, 39, 43.

6. A biological material, wherein the biological material is selected from any one of the following D1) to D3):
D1) a nucleic acid molecule encoding the nanobody according to any one of claim 1 or a nanobody mutants thereof, or a fusion protein comprising the nanobody mutants;
the nanobody mutants is selected from any one of B1) or B2):
B1) the amino acid sequence is shown in SEQ ID NO: 21;
B2) an amino acid sequence obtained by linking a tag or a signal peptide to the N-terminus and/or C-terminus of the amino acid sequence defined in B1);
wherein the fusion protein is obtained by linking the mutant nanobody to an RBCC domain or its mutant via a second linker, the amino acid sequence of the second linker is shown in SEQ ID NO: 31, the amino acid sequence of the RBCC domain is shown in SEQ ID NO: 29, the amino acid sequence of the RBCC domain mutant is shown in SEQ ID NO: 30; and the amino acid sequence of the fusion protein is shown in any one of SEQ ID NOs: 35, 36, 38, 39, 43;
D2) a recombinant vector comprising the nucleic acid molecule of D1);
D3) a recombinant cell comprising the nucleic acid molecule of D1) or the recombinant vector of D2).

7. The biological material according to claim 6, wherein the nanobody further comprises FR1, FR2, FR3 and FR4;
wherein the amino acid sequence of the FR1 is shown in SEQ ID NO: 9, the amino acid sequence of the FR2 is shown in SEQ ID NO: 11, the amino acid sequence of the FR3 is shown in SEQ ID NO: 13, and the amino acid sequence of the FR4 is shown in SEQ ID NO: 15;
or the amino acid sequence of the FR1 is shown in SEQ ID NO: 10, the amino acid sequence of the FR2 is shown in SEQ ID NO: 12, the amino acid sequence of the FR3 is shown in SEQ ID NO: 14, and the amino acid sequence of the FR4 is shown in SEQ ID NO: 16.

8. The biological material according to claim 7, wherein the nanobody is selected from any one of the following:
A1) the amino acid sequence is shown in any one of SEQ ID NOs: 17-20;
A2) an amino acid sequence obtained by linking a tag or a signal peptide to the N-terminus and/or C-terminus of the amino acid sequence defined in A1).

9. A pharmaceutical composition, wherein the pharmaceutical composition comprises any one of the following F1) to F5), and a pharmaceutically acceptable carrier;
F1) the nanobody according to claim 1;
F2) a mutant of the nanobody as defined in F1), wherein the nanobody mutant is selected from any one of the following:
B1) the amino acid sequence is shown in SEQ ID NO: 21;
B2) an amino acid sequence obtained by linking a tag or a signal peptide to the N-terminus and/or C-terminus of the amino acid sequence defined in B1);
F3) a fusion protein obtained by linking the nanobody mutant of F2) with an RBCC domain or a mutant thereof via a second linker;
wherein the amino acid sequence of the second linker is shown in SEQ ID NO: 31, the amino acid sequence of the RBCC domain is shown in SEQ ID NO: 29, the amino acid sequence of the RBCC domain mutant is shown in SEQ ID NO: 30; and the amino acid sequence of the fusion protein is shown in any one of SEQ ID NOs: 35, 36, 38, 39, 43;
F4) a biological material selected from any one of the following D1) to D3):
D1) a nucleic acid molecule encoding the nanobody according to any one of claim 1 or a nanobody mutant thereof, or a fusion protein comprising the nanobody mutants;
the nanobody mutants is selected from any one of B1) or B2):
B1) the amino acid sequence is shown in SEQ ID NO: 21;
B2) an amino acid sequence obtained by linking a tag or a signal peptide to the N-terminus and/or C-terminus of the amino acid sequence defined in B1);
The fusion protein is obtained by linking the nanobody mutants with an RBCC domain or a mutant thereof via a second linker;
wherein the amino acid sequence of the second linker is shown in SEQ ID NO: 31, the amino acid sequence of the RBCC domain is shown in SEQ ID NO: 29, the amino acid sequence of the RBCC domain mutant is shown in SEQ ID NO: 30; and the amino acid sequence of the fusion protein is shown in any one of SEQ ID NOs: 35, 36, 38, 39, 43;
D2) a recombinant vector comprising the nucleic acid molecule of D1);
D3) a recombinant cell comprising the nucleic acid molecule of D1) or the recombinant vector of D2).

10. The pharmaceutical composition according to claim 9, wherein the nanobody further comprises FR1, FR2, FR3 and FR4;
wherein the amino acid sequence of the FR1 is shown in SEQ ID NO: 9, the amino acid sequence of the FR2 is shown in SEQ ID NO: 11, the amino acid sequence of the FR3 is shown in SEQ ID NO: 13, and the amino acid sequence of the FR4 is shown in SEQ ID NO: 15;
or the amino acid sequence of the FR1 is shown in SEQ ID NO: 10, the amino acid sequence of the FR2 is shown in SEQ ID NO: 12, the amino acid sequence of the FR3 is shown in SEQ ID NO: 14, and the amino acid sequence of the FR4 is shown in SEQ ID NO: 16.

11. The pharmaceutical composition according to claim 10, wherein the nanobody is selected from any one of the following A1) or A2):
A1) the amino acid sequence is shown in any one of SEQ ID NOs: 17-20;
A2) an amino acid sequence obtained by linking a tag or a signal peptide to the N-terminus and/or C-terminus of the amino acid sequence defined in A1).

12. A method for preparing the nanobody according to claim 1, or a nanobody mutant thereof, or a fusion protein comprising the nanobody mutants, comprising the steps of:
culturing a recombinant cell to obtain a culture after induced expression;
wherein the recombinant cell comprises a nucleic acid molecule encoding the nanobody, the nanobody mutants or the fusion protein, or a recombinant vector comprising the nucleic acid molecule;
the nanobody mutants is selected from any one of the following:
B1) the amino acid sequence is shown in SEQ ID NO: 21;
B2) an amino acid sequence obtained by linking a tag or a signal peptide to the N-terminus and/or C-terminus of the amino acid sequence defined in B1);
The fusion protein is obtained by linking the nanobody mutants with an RBCC domain or a mutant thereof via a second linker;
wherein the amino acid sequence of the second linker is shown in SEQ ID NO: 31, the amino acid sequence of the RBCC domain is shown in SEQ ID NO: 29, the amino acid sequence of the RBCC domain mutant is shown in SEQ ID NO: 30; and the amino acid sequence of the fusion protein is shown in any one of SEQ ID NOs: 35, 36, 38, 39, 43;
isolating the nanobody, the nanobody mutants or the fusion protein from the culture.

13. The preparation method according to claim 12, wherein the nanobody further comprises FR1, FR2, FR3 and FR4;
wherein the amino acid sequence of the FR1 is shown in SEQ ID NO: 9, the amino acid sequence of the FR2 is shown in SEQ ID NO: 11, the amino acid sequence of the FR3 is shown in SEQ ID NO: 13, and the amino acid sequence of the FR4 is shown in SEQ ID NO: 15;
or the amino acid sequence of the FR1 is shown in SEQ ID NO: 10, the amino acid sequence of the FR2 is shown in SEQ ID NO: 12, the amino acid sequence of the FR3 is shown in SEQ ID NO: 14, and the amino acid sequence of the FR4 is shown in SEQ ID NO: 16.

14. The preparation method according to claim 13, wherein the nanobody is selected from any one of the following:
A1) the amino acid sequence is shown in any one of SEQ ID NOs: 17-20;
A2) an amino acid sequence obtained by linking a tag or a signal peptide to the N-terminus and/or C-terminus of the amino acid sequence defined in A1).


SEQUENCE LISTING (44 sequences)

SEQ ID NO: 1 - protein, 7 aa, synthetic construct
GFNIKDT

SEQ ID NO: 2 - protein, 11 aa, synthetic construct
SGGSEYSYSTF

SEQ ID NO: 3 - protein, 6 aa, synthetic construct
YPTSGY

SEQ ID NO: 4 - protein, 6 aa, synthetic construct
ASMGGL

SEQ ID NO: 5 - protein, 24 aa, synthetic construct
GSGITTIKEMGRSIHEIPREEFDY

SEQ ID NO: 6 - protein, 28 aa, synthetic construct
GSGLVATVKEAGRSIHEIPREELEEFDY

SEQ ID NO: 7 - protein, 18 aa, synthetic construct
GSVGASTLYGSAYEEFDY

SEQ ID NO: 8 - protein, 21 aa, synthetic construct
GLVATVKEAGRSIHEIPREEL

SEQ ID NO: 9 - protein, 26 aa, synthetic construct
MEVQLEESGGGLVQPGGSLRLSCAAS

SEQ ID NO: 10 - protein, 27 aa, synthetic construct
EFQVQLVESGGGSVQAGGSLRLSCTAS

SEQ ID NO: 11 - protein, 19 aa, synthetic construct
YIGWVRQAPGKGEEWVASI

SEQ ID NO: 12 - protein, 19 aa, synthetic construct
SLGWFRQAPGQEREAVAAI

SEQ ID NO: 13 - protein, 41 aa, synthetic construct
TRYADSVKGRFTISADTSKNTAYLQMNSLRAEDTAVYYCAA

SEQ ID NO: 14 - protein, 41 aa, synthetic construct
TYYADSVKGRFTISRDNAKNTVTLQMNNLKPEDTAIYYCAA

SEQ ID NO: 15 - protein, 11 aa, synthetic construct
WGQGTLVTVSS

SEQ ID NO: 16 - protein, 11 aa, synthetic construct
WGQGTQVTVSS

SEQ ID NO: 17 - protein, 134 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGITTIKEMGRSIHEIPREEFDYWGQGTLVTVSS

SEQ ID NO: 18 - protein, 138 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGLVATVKEAGRSIHEIPREELEEFDYWGQGTLVTVSS

SEQ ID NO: 19 - protein, 128 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSVGASTLYGSAYEEFDYWGQGTLVTVSS

SEQ ID NO: 20 - protein, 135 aa, synthetic construct
EFQVQLVESGGGSVQAGGSLRLSCTASGGSEYSYSTFSLGWFRQAPGQEREAVAAIASMGGLTYYADSVKGRFTISRDNAKNTVTLQMNNLKPEDTAIYYCAAGLVATVKEAGRSIHEIPREELWGQGTQVTVSS

SEQ ID NO: 21 - protein, 138 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGLVAQVKEAGRSIHEIPREELEEFDYWGQGTLVTVSS

SEQ ID NO: 22 - DNA, 402 bp, synthetic construct
  source 1..402: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtggcattaccaccattaaagaaatgggccgcagcattcatgaaattccgcgcgaggaatttgactattggggccagggcaccttagttaccgttagctcg

SEQ ID NO: 23 - DNA, 414 bp, synthetic construct
  source 1..414: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtggcctggtggcgaccgtgaaagaagcgggccgcagcattcatgaaattccgcgcgaagaactggaggaatttgactattggggccagggcaccttagttaccgttagctcg

SEQ ID NO: 24 - DNA, 384 bp, synthetic construct
  source 1..384: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtgtgggcgcgagcaccctgtatggtagcgcgtatgaggaatttgactattggggccagggcaccttagttaccgttagctcg

SEQ ID NO: 25 - DNA, 405 bp, synthetic construct
  source 1..405: mol_type = other DNA
gaattccaggttcagctggtggaaagtggcggtggtagtgtgcaggccggcggcagtctgcgtctgagttgtaccgcaagcggtggcagcgaatatagctatagtacctttagcctgggctggtttcgccaggccccgggtcaggaacgcgaagcagttgcagccattgcaagcatgggcggtctgacctattatgcagatagtgttaaaggtcgctttaccattagccgtgataatgccaaaaataccgttaccctgcagatgaataatctgaaaccggaagataccgccatttattattgcgcagcaggcctggtggcgaccgtgaaagaagcgggccgcagcattcatgaaattccgcgcgaagaactgtggggtcagggcacccaggtgaccgtgagcagt

SEQ ID NO: 26 - DNA, 414 bp, synthetic construct
  source 1..414: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtggcctggtggcgcaggtgaaagaagcgggccgcagcattcatgaaattccgcgcgaagaactggaggaatttgactattggggccagggcaccttagttaccgttagctcg

SEQ ID NO: 27 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccgaatttgaaattcgtcataatgtggaagatggcagcgtgcagctggcggatcattatcagcagaataccccgattggcgatggcccagtgctgctgccggatgaccactatctgagcaccgaaagcgtgctgagcaaagatccgaatgaagatcgtgatcatatggtcctgctggaatttgtgaccgcggcaggcattgatctgggcatggatgaactgtataaa

SEQ ID NO: 28 - DNA, 24 bp, synthetic construct
  source 1..24: mol_type = other DNA
ttggaggttttgttccagggtcca

SEQ ID NO: 29 - protein, 234 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRR

SEQ ID NO: 30 - protein, 234 aa, synthetic construct
ASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRR

SEQ ID NO: 31 - protein, 16 aa, synthetic construct
GGGGSYPYDVPDYAGS

SEQ ID NO: 32 - DNA, 702 bp, synthetic construct
  source 1..702: mol_type = other DNA
gcttcagcagcacgcttgacaatgatgtgggaggaggtcacatgccctatctgcctggaccccttcgtggagcctgtgagcatcgagtgtggccacagcttctgccaggaatgcatctctcaggttgggaaaggtgggggcagcgtctgtcctgtgtgccggcagcgctttctgctcaagaatctccggcccaatcgacagctagccaacatggtgaacaaccttaaagaaatcagccaggaggccagagagggcacacagggggaacggtgtgcagtgcatggagagagacttcacctgttctgtgagaaagatgggaaggccctttgctgggtatgtgcccagtctcggaaacaccgtgaccacgccatggtccctcttgaggaggctgcacaggagtaccaggagaagctccaggtggcattaggggaactgagaagaaagcaggagttggctgagaagttggaagtggaaattgcaataaagagagcagactggaagaaaacagtggaaacacagaaatctaggattcacgcagagtttgtgcagcaaaaaaacttcctggttgaagaagaacagaggcagctgcaggagctggagaaggatgagagggagcagctgagaatcctgggggagaaagaggccaagctggcccagcagagccaggccctacaggagctcatctcagagctagatcgaagg

SEQ ID NO: 33 - DNA, 702 bp, synthetic construct
  source 1..702: mol_type = other DNA
gcttcagcagcacgcttgacaatgatgtgggaggaggtcacatgccctatctgcctggaccccttcgtggagcctgtgagcatcgagtgtggccacagcttctgccaggaatgcatctctcaggttgggaaaggtgggggcagcgtctgtcctgtgtgccggcagcgctttctgctcaagaatctccggcccaatcgacagctagccaacatggtgaacaaccttaaagaaatcgaacaggaggccagagagggcacacagggggaacggtgtgcagtgcatggagagagacttcacctgttctgtgagaaagatgggaaggccctttgctgggtatgtgcccagtctcggaaacaccgtgaccacgccatggtccctcttgaggaggctgcacaggagtaccaggagaagctccaggtggcattaggggaactgagaagaaagcaggagttggctgagaagttggaagtggaaattgcaataaagagagcagactggaagaaaacagtggaaacacagaaatctaggattcacgcagagtttgtgcagcaaaaaaacttcctggttgaagaagaacagaggcagctgcaggagctggagaaggatgagagggagcagctgagaatcctgggggagaaagaggccaagctggcccagcagagccaggccctacaggagctcatctcagagctagatcgaagg

SEQ ID NO: 34 - DNA, 48 bp, synthetic construct
  source 1..48: mol_type = other DNA
ggcggaggtggcagctatccgtatgatgttccggattatgcaggaagc

SEQ ID NO: 35 - protein, 398 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGITTIKEMGRSIHEIPREEFDYWGQGTLVTVSS

SEQ ID NO: 36 - protein, 402 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGLVATVKEAGRSIHEIPREELEEFDYWGQGTLVTVSS

SEQ ID NO: 37 - protein, 392 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSVGASTLYGSAYEEFDYWGQGTLVTVSS

SEQ ID NO: 38 - protein, 399 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSEFQVQLVESGGGSVQAGGSLRLSCTASGGSEYSYSTFSLGWFRQAPGQEREAVAAIASMGGLTYYADSVKGRFTISRDNAKNTVTLQMNNLKPEDTAIYYCAAGLVATVKEAGRSIHEIPREELWGQGTQVTVSS

SEQ ID NO: 39 - protein, 402 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGLVAQVKEAGRSIHEIPREELEEFDYWGQGTLVTVSS

SEQ ID NO: 40 - protein, 398 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGITTIKEMGRSIHEIPREEFDYWGQGTLVTVSS

SEQ ID NO: 41 - protein, 402 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGLVATVKEAGRSIHEIPREELEEFDYWGQGTLVTVSS

SEQ ID NO: 42 - protein, 392 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSVGASTLYGSAYEEFDYWGQGTLVTVSS

SEQ ID NO: 43 - protein, 399 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSEFQVQLVESGGGSVQAGGSLRLSCTASGGSEYSYSTFSLGWFRQAPGQEREAVAAIASMGGLTYYADSVKGRFTISRDNAKNTVTLQMNNLKPEDTAIYYCAAGLVATVKEAGRSIHEIPREELWGQGTQVTVSS

SEQ ID NO: 44 - protein, 402 aa, synthetic construct
MDYKDDDDKVPKDPASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSGLVAQVKEAGRSIHEIPREELEEFDYWGQGTLVTVSS